(19) United States
(12) Patent Application Publication
(10) Pub. No.: US 2015/0290261 A1
(43) Pub. Date: Oct. 15, 2015

(54) METHODS OF USE FOR PROBIOTICS AND PREBIOTICS

(71) Applicant: Mead Johnson Nutrition Company, Glenview, IL (US)

(72) Inventors: Maciej Chichlowski, Fair Oaks, CA (US); Brian Berg, Evansville, IN (US); Colin Rudolph, San Francisco, CA (US); Robert J. McMahon, Cincinnati, OH (US); Rosaline Waworuntu, Evansville, IN (US)

(21) Appl. No.: 14/503,930

(22) Filed: Oct. 1, 2014

Related U.S. Application Data
(63) Continuation-in-part of application No. 14/249,548, filed on Apr. 10, 2014.

(51) Int. Cl. A61K 35/747; A23L 1/308; A23L 1/29; A23L 1/30; A61K 31/716; A61K 31/702

(52) U.S. Cl. CPC A61K 35/747; A61K 31/716; A61K 31/702; A23L 1/296; A23L 1/3014; A23L 1/3088; A61K 2035/115

(57) ABSTRACT

The present disclosure relates to method(s) for reducing the risk of visceral pain hypersensitivity, modulating the gut-brain axis, or reducing the local inflammatory response in a subject. The method(s) include providing a nutritional composition that includes Lactobacillus rhamnosus GG (LGG), galacto-oligosaccharide(s) (GOS) and polydextrose (PDX) to the subject. The combination of LGG, GOS, and PDX may exhibit additive or synergistic beneficial health effects when consumed. The nutritional compositions herein are suitable for administration to children and infants.

### TECHNICAL FIELD

The present disclosure relates generally to a method of reducing visceral hyperalgesia and reducing functional abdominal pain (FAP) in a target subject by administering a nutritional composition including a combination of Lactobacillus rhamnosus GG (LGG), galacto-oligosaccharide (GOS) and polydextrose (PDX). The nutritional compositions are suitable for administration to pediatric subjects. Additionally, the disclosure provides methods for modulating the gut-brain axis, supporting early modification of gut microbiota, and reducing local inflammatory response by providing the nutritional compositions disclosed herein. The nutritional composition(s) provided herein including a combination of LGG, GOS, and PDX may provide additive and or/synergistic beneficial health effects.

### BACKGROUND ART

The early neonatal period is a critical time for the development of neural pathways, which require use-dependent activity for normal development. However, abnormal stimuli such as stress, sustained pain, or prolonged inflammation in the neonatal period may adversely affect development and subsequently lead to lower thresholds for pain later in life.

In addition, therapeutic use of antibiotics can cause abnormal development by skewing the microbiome, possibly altering the homeostatic mechanisms or leading to expansion of pathogen reservoir. Further, harmful stimulus in the viscera may lead to long-term visceral hypersensitivity observed during childhood. For example, population-based studies have demonstrated that approximately 8% of children experience recurrent FAP and about 61% of these children continue to report abdominal pain or irritable bowel syndrome-like symptoms in their adulthood. (Chitkara, Rawat et al., 2005).

As such, what is needed are methods for improving gut microbiota composition and activity such that pediatric subjects will experience a lower incidence of visceral hyperalgesia and a lower incidence of digestive tract infections, such that this lower pain threshold can continue on into the subjects adult life. As such, provided herein method of improving the gut microbiota in a target subject, by providing a nutritional composition that includes a combination of LGG, GOS and PDX, to the target subject. Additionally, disclosed herein are methods for lowering the incidence of visceral hyperalgesia, lowering the incidence of digestive tract infections, normalizing colonic permeability, and/or supporting a balanced immune response, by administering a nutritional composition having the specific combination of LGG, GOS, and PDX as described herein.

### BRIEF SUMMARY

Briefly, the present disclosure is directed, in an embodiment, to a method for reducing the risk of visceral pain hypersensitivity and/or lowering the incidence of visceral hyperalgesia in a target subject by providing a nutritional composition that contains a carbohydrate source, a protein source, a fat source, and a combination of LGG, GOS, and PDX. This nutritional composition may further reduce incidents of FAP. In some embodiments, the target subject is a pediatric subject. In some embodiments, the nutritional compositions disclosed herein including the combination of LGG, GOS, and PDX may be in infant formula.

In certain embodiments the nutritional composition(s) may optionally contain a source of long chain polyunsaturated fatty acids (“LCPUFAs”), for example docosahexaenoic acid (“DHA”) and/or arachidonic acid (“ARA”), β-glucan, lactoferrin, a source of iron, and mixtures of one or more thereof.

Additionally, the disclosure is directed to a method of improving gut microbiota composition and/or function by providing to a target subject a nutritional composition having a combination of LGG, GOS, and PDX. Further provided is a method for lowering the incidence of digestive tract infections by providing to a target subject a nutritional composition having a combination of LGG, GOS, and PDX.

The disclosure further provides a method of normalizing colonic permeability by providing to a target subject a nutritional composition having a combination of LGG, GOS, and PDX. Additionally provided are methods of supporting a balanced immunity response and/or anti-inflammatory response by providing to a target subject a nutritional composition having a combination of LGG, GOS, and PDX. Further disclosed herein are methods of reducing the incidence of infantile colic by providing to a target subject a nutritional composition having a combination of LGG, GOS and PDX.

It is to be understood that both the foregoing general description and the following detailed description present embodiments of the disclosure and are intended to provide an overview or framework for understanding the nature and character of the disclosure as it is claimed. The description serves to explain the principles and operations of the claimed subject matter. Other and further features and advantages of the present disclosure will be readily apparent to those skilled in the art upon a reading of the following disclosure.

### BRIEF DESCRIPTION OF THE DRAWINGS

FIG. 1 illustrates the effects of PDX/GOS and PDX/GOS+LGG on gut microbiota at the genus level.

FIG. 2 illustrates the effects of PDX/GOS and PDX/GOS+LGG on gut microbiota at the genus level.

FIG. 3 illustrates the effect on bacterial diversity by PDX/GOS and PDX/GOS+LGG.

FIG. 4 illustrates the effects of PDX/GOS and PDX/GOS+LGG on gut microbiota at the phylum level.

FIG. 5 illustrates the results of the novel object recognition test of rats fed PDX/GOS.

FIG. 6A illustrates the effect of LGG treatment on the levels of neurotransmitters in the brain stem and subcortex of control and experimental rats.

FIG. 6B illustrates the effect of LGG treatment on the levels of neurotransmitters in the brain stem and subcortex of control and experimental rats.

FIG. 7 illustrates the effect of LGG on the viscera-motor response (“VMR”) to colorectal distension (“CRD”) in neonatal colitis rats.

### DETAILED DESCRIPTION

Reference now will be made in detail to the embodiments of the present disclosure, one or more examples of which are set forth hereinbelow. Each example is provided by way of explanation of the nutritional composition of the present disclosure and is not a limitation. In fact, it will be apparent to those skilled in the art that various modifications and variations can be made to the teachings of the present disclosure without departing from the scope of the disclosure. For instance, features illustrated or described as part of one embodiment, can be used with another embodiment to yield a still further embodiment.

Thus, it is intended that the present disclosure covers such modifications and variations as come within the scope of the appended claims and their equivalents. Other objects, features and aspects of the present disclosure are disclosed in or are apparent from the following detailed description. It is to be understood by one of ordinary skill in the art that the present discussion is a description of exemplary embodiments only and is not intended as limiting the broader aspects of the present disclosure.

The present disclosure relates generally to methods of improving gut microbiota composition and/or activity, lowering the incidence of visceral hyperalgeisa, lowering the incidence of digestive tract infection, normalizing colonic permeability, and/or supporting a balanced immune response by providing a nutritional composition that includes a combination of LGG, GOS, and PDX.

“Nutritional composition” means a substance or formulation that satisfies at least a portion of a subject's nutrient requirements. The terms “nutritional(s)”, “nutritional formula(s)”, “enteral nutritional(s)”, and “nutritional supplement(s)” are used as non-limiting examples of nutritional composition(s) throughout the present disclosure. Moreover, “nutritional composition(s)” may refer to liquids, powders, gels, pastes, solids, concentrates, suspensions, or ready-to-use forms of enteral formulas, oral formulas, formulas for infants, formulas for pediatric subjects, formulas for children, growing-up milks and/or formulas for adults.

“Pediatric subject” means a human less than 13 years of age. In some embodiments, a pediatric subject refers to a human subject that is between birth and 8 years old. In other embodiments, a pediatric subject refers to a human subject between 1 and 6 years of age. In still further embodiments, a pediatric subject refers to a human subject between 6 and 12 years of age. The term “pediatric subject” may refer to infants (preterm or fullterm) and/or children, as described below.

“Infant” means a human subject ranging in age from birth to not more than one year and includes infants from 0 to 12 months corrected age. The phrase “corrected age” means an infant's chronological age minus the amount of time that the infant was born premature. Therefore, the corrected age is the age of the infant if it had been carried to full term. The term infant includes low birth weight infants, very low birth weight infants, and preterm infants. “Preterm” means an infant born before the end of the 37th week of gestation. “Full term” means an infant born after the end of the 37th week of gestation.

“Child” means a subject ranging in age from 12 months to about 13 years. In some embodiments, a child is a subject between the ages of 1 and 12 years old. In other embodiments, the terms “children” or “child” refer to subjects that are between one and about six years old, or between about seven and about 12 years old. In other embodiments, the terms “children” or “child” refer to any range of ages between 12 months and about 13 years.

“Infant formula” means a composition that satisfies at least a portion of the nutrient requirements of an infant. In the United States, the content of an infant formula is dictated by the federal regulations set forth at 21 C.F.R. Sections 100, 106, and 107. These regulations define macronutrient, vitamin, mineral, and other ingredient levels in an effort to simulate the nutritional and other properties of human breast milk.

The term “growing-up milk” refers to a broad category of nutritional compositions intended to be used as a part of a diverse diet in order to support the normal growth and development of a child between the ages of about 1 and about 6 years of age.

“Nutritionally complete” means a composition that may be used as the sole source of nutrition, which would supply essentially all of the required daily amounts of vitamins, minerals, and/or trace elements in combination with proteins, carbohydrates, and lipids. Indeed, “nutritionally complete” describes a nutritional composition that provides adequate amounts of carbohydrates, lipids, essential fatty acids, proteins, essential amino acids, conditionally essential amino acids, vitamins, minerals and energy required to support normal growth and development of a subject.

A nutritional composition that is “nutritionally complete” for a full term infant will, by definition, provide qualitatively and quantitatively adequate amounts of all carbohydrates, lipids, essential fatty acids, proteins, essential amino acids, conditionally essential amino acids, vitamins, minerals, and energy required for growth of the full term infant.

A nutritional composition that is “nutritionally complete” for a child will, by definition, provide qualitatively and quantitatively adequate amounts of all carbohydrates, lipids, essential fatty acids, proteins, essential amino acids, conditionally essential amino acids, vitamins, minerals, and energy required for growth of a child.

The nutritional composition of the present disclosure may be substantially free of any optional or selected ingredients described herein, provided that the remaining nutritional composition still contains all of the required ingredients or features described herein. In this context, and unless otherwise specified, the term “substantially free” means that the selected composition may contain less than a functional amount of the optional ingredient, typically less than 0.1% by weight, and also, including zero percent by weight of such optional or selected ingredient.

Therefore, a nutritional composition that is “nutritionally complete” for a preterm infant will, by definition, provide qualitatively and quantitatively adequate amounts of carbohydrates, lipids, essential fatty acids, proteins, essential amino acids, conditionally essential amino acids, vitamins, minerals, and energy required for growth of the preterm infant.

A nutritional composition that is “nutritionally complete” for a full term infant will, by definition, provide qualitatively and quantitatively adequate amounts of all carbohydrates, lipids, essential fatty acids, proteins, essential amino acids, conditionally essential amino acids, vitamins, minerals, and energy required for growth of the full term infant.

A nutritional composition that is “nutritionally complete” for a child will, by definition, provide qualitatively and quantitatively adequate amounts of all carbohydrates, lipids, essential fatty acids, proteins, essential amino acids, conditionally essential amino acids, vitamins, minerals, and energy required for growth of a child.

As applied to nutrients, the term “essential” refers to any nutrient that cannot be synthesized by the body in amounts sufficient for normal growth and to maintain health and that, therefore, must be supplied by the diet. The term “conditionally essential” as applied to nutrients means that the nutrient must be supplied by the diet under conditions when adequate amounts of the precursor compound is unavailable to the body for endogenous synthesis to occur.

The term “degree of hydrolysis” refers to the extent to which peptide bonds are broken by a hydrolysis method. For example, the protein equivalent source of the present disclosure may, in some embodiments comprise hydrolyzed protein having a degree of hydrolysis of no greater than 40%. For this example, this means that at least 40% of the total peptide bonds have been cleaved by a hydrolysis method.

The term “partially hydrolyzed” means having a degree of hydrolysis which is greater than 0% but less than 50%.

The term “extensively hydrolyzed” means having a degree of hydrolysis which is greater than or equal to 50%.

“Probiotic” means a microorganism with low or no pathogenicity that exerts at least one beneficial effect on the health of the host. An example of a probiotic is LGG.

In an embodiment, the probiotic(s) may be viable or non-viable. As used herein, the term “viable”, refers to live microorganisms. The term “non-viable” or “non-viable probiotic” means non-living probiotic microorganisms, their cellular components and/or metabolites thereof. Such non-viable probiotics may have been heat-killed or otherwise inactivated, but they retain the ability to favorably influence the health of the host. The probiotics useful in the present disclosure may be naturally-occurring, synthetic or developed through the genetic manipulation of organisms, whether such source is now known or later developed.

The term “inactivated probiotic” means a probiotic wherein the metabolic activity or reproductive ability of the referenced probiotic organism has been reduced or destroyed. The “inactivated probiotic” does, however, still retain, at the cellular level, at least a portion its biological glycol-protein and DNA/RNA structure. As used herein, the term “inactivated” is synonymous with “non-viable”. More specifically, a non-limiting example of an inactivated probiotic is inactivated Lactobacillus rhamnosus GG (“LGG”) or “inactivated LGG”.

The term “cell equivalent” refers to the level of non-viable, non-replicating probiotics equivalent to an equal number of viable cells. The term “non-replicating” is to be understood as the amount of non-replicating microorganisms obtained from the same amount of replicating bacteria (cfu/g), including inactivated probiotics, fragments of DNA, cell wall or cytoplasmic compounds. In other words, the quantity of non-living, non-replicating organisms is expressed in terms of cfu as if all the microorganisms were alive, regardless whether they are dead, non-replicating, inactivated, fragmented etc.

“Prebiotic” means a non-digestible food ingredient that beneficially affects the host by selectively stimulating the growth and/or activity of one or a limited number of bacteria in the digestive tract that can improve the health of the host. Examples of prebiotics include PDX and GOS.

“β-glucan” means all β-glucan, including specific types of β-glucan, such as β-1,3-glucan or β-1,3;1,6-glucan. Moreover, β-1,3;1,6-glucan is a type of β-1,3-glucan. Therefore, the term “β-1,3-glucan” includes β-1,3;1,6-glucan.

As used herein, “non-human lactoferrin” means lactoferrin which is produced by or obtained from a source other than human breast milk. In some embodiments, non-human lactoferrin is lactoferrin that has an amino acid sequence that is different than the amino acid sequence of human lactoferrin. In other embodiments, non-human lactoferrin for use in the present disclosure includes human lactoferrin produced by a genetically modified organism. The term “organism”, as used herein, refers to any contiguous living system, such as animal, plant, fungus or micro-organism.

“Inherent lutein” or “lutein from endogenous sources” refers to any lutein present in the formulas that is not added as such, but is present in other components or ingredients of the formulas; the lutein is naturally present in such other components.

All percentages, parts and ratios as used herein are by weight of the total composition, unless otherwise specified.

All references to singular characteristics or limitations of the present disclosure shall include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The methods and compositions of the present disclosure, including components thereof, can comprise, consist of, or consist essentially of the essential elements and limitations of the embodiments described herein, as well as any additional or optional ingredients, components or limitations described herein or otherwise useful in nutritional compositions.

As used herein, the term “about” should be construed to refer to both of the numbers specified as the endpoint(s) of any range. Any reference to a range should be considered as providing support for any subset within that range.

The present disclosure is directed to a method of improving gut microbiota composition and activity by providing a nutritional composition including a combination of LGG, GOS, and PDX.

Briefly, without being bound by any particular theory, the effect on visceral hyperalgesia of lactobacillus strains in combination with PDX and GOS may be synergistic as compared to when these nutrients are administered individually.

Without being bound by any particular theory, there are numerous physiological and biochemical mechanisms through which the nutritional composition, including LGG, could positively influence the actions of the gut-brain axis. For example, gastrointestinal health could be influenced by affecting the immune system and peripheral nervous system through cytokines and other mediators that promote afferent sensitization as a probable cause for the development of post-inflammatory visceral hypersensitivity. Further, the nutritional composition could cause the displacement of gas producing, bile salt-deconjugating bacteria strains.

Potential mechanisms of action for the nutritional composition disclosed herein include, but are not limited to: promotion of a microbiological environment (e.g. acidification, modified lactic and/or short-chain fatty acid profiles, increased antimicrobials) that competitively exclude pro-inflammatory bacteria (e.g. Enterobacteriaceae, etc.) and/or bacteria (e.g. Clostridium perfringens, Clostridium difficile) that produce inflammatory or neurotoxic substances (e.g. endotoxin and epsilon toxin, respectively); alleviate symptoms of gastrointestinal inflammation (e.g. pain/discomfort, bloating/distension), and normalizing the ratio of anti-inflammatory/pro-inflammatory cytokines (IL-10/IL-12), for example, stimulating the anti-inflammatory cytokine, IL-10, production through interaction with Toll-Like Receptors (e.g. TLR2) and/or other Pattern-Recognition Receptors (PRR) carried by dendritic and/or other immune cells; biosynthesis of neurotransmitters (e.g. glutamate) and/or neurotransmitter precursors (e.g. tryptophan); biosynthesis of nutrients/micronutrients associated with neurological development/processes (e.g. folic acid, choline, glutamine, iron, zinc, etc.); amelioration of stress-induces alterations in neurological development/processes (e.g. corticotrophin-releasing factor, etc.); reduction of post-inflammatory hypersensitivity via normalization of serotonin (5-HT) receptors; and secretion of factors that directly improve colonic mucosal integrity, transepithelial resistance, decrease inflammation, reduce mannitol flux and increase expression of tight junction proteins.

Accordingly, as provided herein, the specific combination of probiotic material and prebiotic material, in combination, may optimize the composition of gastrointestinal microbiota and support development of the gut-brain axis in pediatric subjects, including infants and children. Moreover, the specific combination of probiotics and prebiotics described herein may lower visceral hyperalgesia and FAP in pediatric subjects, including infants and children, when administered to the pediatric subject.

In some embodiments the nutritional composition comprises Lactobacillus rhamnosus GG (ATCC number 53103). In some embodiments, the disclosed nutritional composition(s) described herein may also comprise a source of probiotic other than LGG. Additional probiotics that may be included in the nutritional composition include, but are not limited to: Bifidobacterium species, Bifidobacterium longum BB536 (BL999, ATCC: BAA-999), Bifidobacterium longum AH1206 (NCIMB: 41382), Bifidobacterium breve AH1205 (NCIMB: 41387), Bifidobacterium infantis 35624 (NCIMB: 41003), and Bifidobacterium animalis subsp. lactis BB-12 (DSM No. 10140) or any combination thereof.

In some embodiments, the nutritional composition includes LGG in an amount of from about 1×10⁴ cfu/100 kcal to about 1.5×10¹⁰ cfu/100 kcal. In other embodiments, the nutritional composition comprises LGG in an amount of from about 1×10⁶ cfu/100 kcal to about 1×10⁹ cfu/100 kcal. Still, in certain embodiments, the nutritional composition may include LGG in an amount of from about 1×10⁷ cfu/100 kcal to about 1×10⁸ cfu/100 kcal. In some embodiments, where LGG is not included at the upper limit of the concentration range, additional probiotics may be included up to the upper limit concentration specified.

In some embodiments, the nutritional composition includes a culture supernatant from a late-exponential growth phase of a probiotic batch-cultivation process, as disclosed in international published application no. WO 2013/142403, which is hereby incorporated by reference in its entirety. Without wishing to be bound by theory, it is believed that the activity of the culture supernatant can be attributed to the mixture of components (including proteinaceous materials, and possibly including (exo)polysaccharide materials) as found released into the culture medium at a late stage of the exponential (or “log”) phase of batch cultivation of the probiotic. The term “culture supernatant” as used herein, includes the mixture of components found in the culture medium. The stages recognized in batch cultivation of bacteria are known to the skilled person. These are the “lag,” the “log” (“logarithmic” or “exponential”), the “stationary” and the “death” (or “logarithmic decline”) phases. In all phases during which live bacteria are present, the bacteria metabolize nutrients from the media, and secrete (exert, release) materials into the culture medium. The composition of the secreted material at a given point in time of the growth stages is not generally predictable.

In an embodiment, a culture supernatant is obtainable by a process comprising the steps of (a) subjecting a probiotic such as LGG to cultivation in a suitable culture medium using a batch process; (b) harvesting the culture supernatant at a late exponential growth phase of the cultivation step, which phase is defined with reference to the second half of the time between the lag phase and the stationary phase of the batch-cultivation process; (c) optionally removing low molecular weight constituents from the supernatant so as to retain molecular weight constituents above 5-6 kiloDaltons (kDa); (d) removing liquid contents from the culture supernatant so as to obtain the composition.

The culture supernatant may comprise secreted materials that are harvested from a late exponential phase. The late exponential phase occurs in time after the mid exponential phase (which is halftime of the duration of the exponential phase, hence the reference to the late exponential phase as being the second half of the time between the lag phase and the stationary phase). In particular, the term “late exponential phase” is used herein with reference to the latter quarter portion of the time between the lag phase and the stationary phase of the LGG batch-cultivation process. In some embodiments, the culture supernatant is harvested at a point in time of 75% to 85% of the duration of the exponential phase, and may be harvested at about ⅚ of the time elapsed in the exponential phase.

In some embodiments, the nutritional composition comprises the culture supernatant from about 0.015 mg/100 kcal to about 1.5 mg/100 kcal. In some embodiments, where the nutritional composition does not include LGG at the upper limit of the concentration ranges disclosed herein, the nutritional composition may further comprise a culture supernatant.

The disclosed nutritional composition also comprise a source of prebiotics, specifically GOS and PDX. In some embodiments, the amount of GOS in the nutritional composition may be from about 0.1 g/100 kcal to about 1.5 g/100 kcal. In another embodiment, the amount of GOS in the nutritional composition may be from about 0.15 g/100 kcal to about 0.5 g/100 kcal.

The amount of PDX in the nutritional composition may, in some embodiments, be within the range of from about 0.1 g/100 kcal to about 0.5 g/100 kcal. In a particular embodiment, GOS and PDX are supplemented into the nutritional composition in a total amount of about at least about 0.2 g/100 kcal and can be about 0.2 g/100 kcal to about 1.5 g/100 kcal. In some embodiments, the nutritional composition may comprise GOS and PDX in a total amount of from about 0.6 to about 0.8 g/100 kcal.

In some embodiments, the nutritional composition may include prebiotics, in addition to GOS and PDX. In some embodiments, additional prebiotics useful in the present disclosure may include: lactulose, lactosucrose, raffinose, gluco-oligosaccharide, inulin, fructo-oligosaccharide, isomalto-oligosaccharide, soybean oligosaccharides, lactosucrose, xylo-oligosaccharide, chito-oligosaccharide, manno-oligosaccharide, aribino-oligosaccharide, siallyl-oligosaccharide, fuco-oligosaccharide, and gentio-oligosaccharides. In embodiments where GOS and PDX are not included at the upper limit of their respective concentration range, additional prebiotics may be included up to the upper limit concentration specified.

In one embodiment, where the nutritional composition is an infant formula, the combination of LGG, GOS, and PDX may be added to a commercially available infant formula. For example, Enfalac, Enfamil®, Enfamil® Premature Formula, Enfamil® with Iron, Enfamil® LIPIL®, Lactofree®, Nutramigen®, Pregestimil®, and ProSobee® (available from Mead Johnson & Company, Evansville, Ind., U.S.A.) may be supplemented with LGG, GOS, and PDX, and used in practice of the current disclosure.

The nutritional composition(s) of the present disclosure may also comprise a carbohydrate source. Carbohydrate sources can be any used in the art, e.g., lactose, glucose, fructose, corn syrup solids, maltodextrins, sucrose, starch, rice syrup solids, and the like. The amount of carbohydrate in the nutritional composition typically can vary from between about 5 g and about 25 g/100 kcal. In some embodiments, the amount of carbohydrate is between about 6 g and about 22 g/100 kcal. In other embodiments, the amount of carbohydrate is between about 12 g and about 14 g/100 kcal. In some embodiments, corn syrup solids are preferred. Moreover, hydrolyzed, partially hydrolyzed, and/or extensively hydrolyzed carbohydrates may be desirable for inclusion in the nutritional composition due to their easy digestibility. Specifically, hydrolyzed carbohydrates are less likely to contain allergenic epitopes.

Non-limiting examples of carbohydrate materials suitable for use herein include hydrolyzed or intact, naturally or chemically modified, starches sourced from corn, tapioca, rice or potato, in waxy or non-waxy forms. Non-limiting examples of suitable carbohydrates include various hydrolyzed starches characterized as hydrolyzed cornstarch, maltodextrin, maltose, corn syrup, dextrose, corn syrup solids, glucose, and various other glucose polymers and combinations thereof. Non-limiting examples of other suitable carbohydrates include those often referred to as sucrose, lactose, fructose, high fructose corn syrup, indigestible oligosaccharides such as fructooligosaccharides and combinations thereof.

The nutritional composition(s) of the disclosure may also comprise a protein source. The protein source can be any used in the art, e.g., nonfat milk, whey protein, casein, soy protein, hydrolyzed protein, amino acids, and the like. Bovine milk protein sources useful in practicing the present disclosure include, but are not limited to, milk protein powders, milk protein concentrates, milk protein isolates, nonfat milk solids, nonfat milk, nonfat dry milk, whey protein, whey protein isolates, whey protein concentrates, sweet whey, acid whey, casein, acid casein, caseinate (e.g. sodium caseinate, sodium calcium caseinate, calcium caseinate) and any combinations thereof.

In one embodiment, the proteins of the nutritional composition are provided as intact proteins. In other embodiments, the proteins are provided as a combination of both intact proteins and partially hydrolyzed proteins, with a degree of hydrolysis of between about 4% and 10%. In certain other embodiments, the proteins are more completely hydrolyzed. In still other embodiments, the protein source comprises amino acids. In yet another embodiment, the protein source may be supplemented with glutamine-containing peptides.

In a particular embodiment of the nutritional composition, the whey:casein ratio of the protein source is similar to that found in human breast milk. In an embodiment, the protein source comprises from about 40% to about 90% whey protein and from about 10% to about 60% casein.

In some embodiments, the nutritional composition comprises between about 1 g and about 7 g of a protein source per 100 kcal. In other embodiments, the nutritional composition comprises between about 3.5 g and about 4.5 g of protein per 100 kcal.

In some embodiments, the nutritional composition described herein comprises a fat source. Appropriate fat sources include, but are not limited to, animal sources, e.g., milk fat, butter, butter fat, egg yolk lipid; marine sources, such as fish oils, marine oils, single cell oils; vegetable and plant oils, such as corn oil, canola oil, sunflower oil, soybean oil, palm olein oil, coconut oil, high oleic sunflower oil, evening primrose oil, rapeseed oil, olive oil, flaxseed (linseed) oil, cottonseed oil, high oleic safflower oil, palm stearin, palm kernel oil, wheat germ oil; medium chain triglyceride oils and emulsions and esters of fatty acids; and any combinations thereof.

In some embodiments, the nutritional composition comprises between about 1 g and about 10 g of a fat source per 100 kcal. In other embodiments, the nutritional composition comprises between about 3.5 g and about 7 g of a fat source per 100 kcal.

In some embodiments the nutritional composition may also include a source of LCPUFAs. In one embodiment the amount of LCPUFA in the nutritional composition is advantageously at least about 5 mg/100 kcal, and may vary from about 5 mg/100 kcal to about 100 mg/100 kcal, more preferably from about 10 mg/100 kcal to about 50 mg/100 kcal. Non-limiting examples of LCPUFAs include, but are not limited to, DHA, ARA, linoleic (18:2 n-6), γ-linolenic (18:3 n-6), dihomo-γ-linolenic (20:3 n-6) acids in the n-6 pathway, α-linolenic (18:3 n-3), stearidonic (18:4 n-3), eicosatetraenoic (20:4 n-3), eicosapentaenoic (20:5 n-3), and docosapentaenoic (22:6 n-3).

In some embodiments, the LCPUFA included in the nutritional composition may comprise DHA. In one embodiment the amount of DHA in the nutritional composition is from about 15 mg/100 kcal to about 75 mg/100 kcal. Still in some embodiments, the amount of DHA in the nutritional composition is from about 10 mg/100 kcal to about 50 mg/100 kcal.

In another embodiment, especially if the nutritional composition is an infant formula, the nutritional composition is supplemented with both DHA and ARA. In this embodiment, the weight ratio of ARA:DHA may be between about 1:3 and about 9:1. In a particular embodiment, the ratio of ARA:DHA is from about 1:2 to about 4:1.

The DHA and ARA can be in natural form, provided that the remainder of the LCPUFA source does not result in any substantial deleterious effect on the infant. Alternatively, the DHA and ARA can be used in refined form.

The disclosed nutritional composition described herein can, in some embodiments, also comprise a source of β-glucan. Glucans are polysaccharides, specifically polymers of glucose, which are naturally occurring and may be found in cell walls of bacteria, yeast, fungi, and plants. Beta glucans (β-glucans) are themselves a diverse subset of glucose polymers, which are made up of chains of glucose monomers linked together via beta-type glycosidic bonds to form complex carbohydrates.

β-1,3-glucans are carbohydrate polymers purified from, for example, yeast, mushroom, bacteria, algae, or cereals. The chemical structure of β-1,3-glucan depends on the source of the β-1,3-glucan. Moreover, various physiochemical parameters, such as solubility, primary structure, molecular weight, and branching, play a role in biological activities of β-1,3-glucans. (Yadomae T., Structure and biological activities of fungal beta-1,3-glucans. Yakugaku Zasshi. 2000; 120:413-431.)

β-1,3-glucans are naturally occurring polysaccharides, with or without β-1,6-glucose side chains that are found in the cell walls of a variety of plants, yeasts, fungi and bacteria. β-1,3;1,6-glucans are those containing glucose units with (1,3) links having side chains attached at the (1,6) position(s). β-1,3;1,6 glucans are a heterogeneous group of glucose polymers that share structural commonalities, including a backbone of straight chain glucose units linked by a β-1,3 bond with β-1,6-linked glucose branches extending from this backbone. While this is the basic structure for the presently described class of β-glucans, some variations may exist. For example, certain yeast β-glucans have additional regions of β(1,3) branching extending from the β(1,6) branches, which add further complexity to their respective structures.

β-glucans derived from baker's yeast, Saccharomyces cerevisiae, are made up of chains of D-glucose molecules connected at the 1 and 3 positions, having side chains of glucose attached at the 1 and 6 positions. Yeast-derived β-glucan is an insoluble, fiber-like, complex sugar having the general structure of a linear chain of glucose units with a β-1,3 backbone interspersed with β-1,6 side chains that are generally 6-8 glucose units in length. More specifically, β-glucan derived from baker's yeast is poly-(1,6)-β-D-glucopyranosyl-(1,3)-β-D-glucopyranose.

Furthermore, β-glucans are well tolerated and do not produce or cause excess gas, abdominal distension, bloating or diarrhea in pediatric subjects. Addition of β-glucan to a nutritional composition for a pediatric subject, such as an infant formula, a growing-up milk or another children's nutritional product, will improve the subject's immune response by increasing resistance against invading pathogens and therefore maintaining or improving overall health.

In some embodiments, the β-glucan is β-1,3;1,6-glucan. In some embodiments, the β-1,3;1,6-glucan is derived from baker's yeast. The nutritional composition may comprise whole glucan particle β-glucan, particulate β-glucan, PGG-glucan (poly-1,6-β-D-glucopyranosyl-1,3-β-D-glucopyranose) or any mixture thereof.

In some embodiments, the amount of β-glucan in the nutritional composition is between about 3 mg and about 17 mg per 100 kcal. In another embodiment the amount of β-glucan is between about 6 mg and about 17 mg per 100 kcal.

The nutritional composition of the present disclosure, may comprise lactoferrin. Lactoferrins are single chain polypeptides of about 80 kD containing 1-4 glycans, depending on the species. The 3-D structures of lactoferrin of different species are very similar, but not identical. Each lactoferrin comprises two homologous lobes, called the N- and C-lobes, referring to the N-terminal and C-terminal part of the molecule, respectively. Each lobe further consists of two sub-lobes or domains, which form a cleft where the ferric ion (Fe3+) is tightly bound in synergistic cooperation with a (bi)carbonate anion. These domains are called N1, N2, C1 and C2, respectively. The N-terminus of lactoferrin has strong cationic peptide regions that are responsible for a number of important binding characteristics. Lactoferrin has a very high isoelectric point (˜pl 9) and its cationic nature plays a major role in its ability to defend against bacterial, viral, and fungal pathogens. There are several clusters of cationic amino acids residues within the N-terminal region of lactoferrin mediating the biological activities of lactoferrin against a wide range of microorganisms.

Lactoferrin for use in the present disclosure may be, for example, isolated from the milk of a non-human animal or produced by a genetically modified organism. The nutritional compositions described herein can, in some embodiments comprise non-human lactoferrin, non-human lactoferrin produced by a genetically modified organism and/or human lactoferrin produced by a genetically modified organism.

Suitable non-human lactoferrins for use in the present disclosure include, but are not limited to, those having at least 48% homology with the amino acid sequence of human lactoferrin. For instance, bovine lactoferrin (“bLF”) has an amino acid composition which has about 70% sequence homology to that of human lactoferrin. In some embodiments, the non-human lactoferrin has at least 65% homology with human lactoferrin and in some embodiments, at least 75% homology. Non-human lactoferrins acceptable for use in the present disclosure include, without limitation, bLF, porcine lactoferrin, equine lactoferrin, buffalo lactoferrin, goat lactoferrin, murine lactoferrin and camel lactoferrin.

bLF suitable for the present disclosure may be produced by any method known in the art. For example, in U.S. Pat. No. 4,791,193, incorporated by reference herein in its entirety, Okonogi et al. discloses a process for producing bovine lactoferrin in high purity. Generally, the process as disclosed includes three steps. Raw milk material is first contacted with a weakly acidic cationic exchanger to absorb lactoferrin followed by the second step where washing takes place to remove nonabsorbed substances. A desorbing step follows where lactoferrin is removed to produce purified bovine lactoferrin. Other methods may include steps as described in U.S. Pat. Nos. 7,368,141, 5,849,885, 5,919,913 and 5,861,491, the disclosures of which are all incorporated by reference in their entirety.

In certain embodiments, lactoferrin utilized in the present disclosure may be provided by an expanded bed absorption (“EBA”) process for isolating proteins from milk sources. EBA, also sometimes called stabilized fluid bed adsorption, is a process for isolating a milk protein, such as lactoferrin, from a milk source comprises establishing an expanded bed adsorption column comprising a particulate matrix, applying a milk source to the matrix, and eluting the lactoferrin from the matrix with an elution buffer comprising about 0.3 to about 2.0 M sodium chloride. Any mammalian milk source may be used in the present processes, although in particular embodiments, the milk source is a bovine milk source. The milk source comprises, in some embodiments, whole milk, reduced fat milk, skim milk, whey, casein, or mixtures thereof.

In particular embodiments, the target protein is lactoferrin, though other milk proteins, such as lactoperoxidases or lactalbumins, also may be isolated. In some embodiments, the process comprises the steps of establishing an expanded bed adsorption column comprising a particulate matrix, applying a milk source to the matrix, and eluting the lactoferrin from the matrix with about 0.3 to about 2.0M sodium chloride. In other embodiments, the lactoferrin is eluted with about 0.5 to about 1.0 M sodium chloride, while in further embodiments, the lactoferrin is eluted with about 0.7 to about 0.9 M sodium chloride.

The expanded bed adsorption column can be any known in the art, such as those described in U.S. Pat. Nos. 7,812,138, 6,620,326, and 6,977,046, the disclosures of which are hereby incorporated by reference herein. In some embodiments, a milk source is applied to the column in an expanded mode, and the elution is performed in either expanded or packed mode. In particular embodiments, the elution is performed in an expanded mode. For example, the expansion ratio in the expanded mode may be about 1 to about 3, or about 1.3 to about 1.7. EBA technology is further described in international published application nos. WO 92/00799, WO 02/18237, WO 97/17132, which are hereby incorporated by reference in their entireties.

The isoelectric point of lactoferrin is approximately 8.9. Prior EBA methods of isolating lactoferrin use 200 mM sodium hydroxide as an elution buffer. Thus, the pH of the system rises to over 12, and the structure and bioactivity of lactoferrin may be comprised, by irreversible structural changes. It has now been discovered that a sodium chloride solution can be used as an elution buffer in the isolation of lactoferrin from the EBA matrix. In certain embodiments, the sodium chloride has a concentration of about 0.3 M to about 2.0 M. In other embodiments, the lactoferrin elution buffer has a sodium chloride concentration of about 0.3 M to about 1.5 M, or about 0.5 m to about 1.0 M.

The lactoferrin that is used in certain embodiments may be any lactoferrin isolated from whole milk and/or having a low somatic cell count, wherein “low somatic cell count” refers to a somatic cell count less than 200,000 cells/mL. By way of example, suitable lactoferrin is available from Tatua Co-operative Dairy Co. Ltd., in Morrinsville, New Zealand, from FrieslandCampina Domo in Amersfoort, Netherlands or from Fonterra Co-Operative Group Limited in Auckland, New Zealand.

Surprisingly, lactoferrin included herein maintains certain bactericidal activity even if exposed to a low pH (i.e., below about 7, and even as low as about 4.6 or lower) and/or high temperatures (i.e., above about 65° C., and as high as about 120° C.), conditions which would be expected to destroy or severely limit the stability or activity of human lactoferrin. These low pH and/or high temperature conditions can be expected during certain processing regimen for nutritional compositions of the types described herein, such as pasteurization. Therefore, even after processing regimens, lactoferrin has bactericidal activity against undesirable bacterial pathogens found in the human gut.

The nutritional composition may, in some embodiments, comprise lactoferrin in an amount from about 10 mg/100 kcal to about 250 mg/100 kcal. In some embodiments, lactoferrin may be present in an amount of from about 50 mg/100 kcal to about 175 mg/100 kcal. Still in some embodiments, lactoferrin may be present in an amount of from about 100 mg/100 kcal to about 150 mg/100 kcal.

The disclosed nutritional composition described herein, can, in some embodiments also comprise an effective amount of iron. The iron may comprise encapsulated iron forms, such as encapsulated ferrous fumarate or encapsulated ferrous sulfate or less reactive iron forms, such as ferric pyrophosphate or ferric orthophosphate.

One or more vitamins and/or minerals may also be added in to the nutritional composition in amounts sufficient to supply the daily nutritional requirements of a subject. It is to be understood by one of ordinary skill in the art that vitamin and mineral requirements will vary, for example, based on the age of the child. For instance, an infant may have different vitamin and mineral requirements than a child between the ages of one and thirteen years. Thus, the embodiments are not intended to limit the nutritional composition to a particular age group but, rather, to provide a range of acceptable vitamin and mineral components.

In embodiments providing a nutritional composition for a child, the composition may optionally include, but is not limited to, one or more of the following vitamins or derivations thereof: vitamin B₁ (thiamin, thiamin pyrophosphate, TPP, thiamin triphosphate, TTP, thiamin hydrochloride, thiamin mononitrate), vitamin B₂ (riboflavin, flavin mononucleotide, FMN, flavin adenine dinucleotide, FAD, lactoflavin, ovoflavin), vitamin B3 (niacin, nicotinic acid, nicotinamide, niacinamide, nicotinamide adenine dinucleotide, NAD, nicotinic acid mononucleotide, NicMN, pyridine-3-carboxylic acid), vitamin B₃-precursor tryptophan, vitamin B₆ (pyridoxine, pyridoxal, pyridoxamine, pyridoxine hydrochloride), pantothenic acid (pantothenate, panthenol), folate (folic acid, folacin, pteroylglutamic acid), vitamin B₁₂ (cobalamin, methylcobalamin, deoxyadenosylcobalamin, cyanocobalamin, hydroxycobalamin, adenosylcobalamin), biotin, vitamin C (ascorbic acid), vitamin A (retinol, retinyl acetate, retinyl palmitate, retinyl esters with other long-chain fatty acids, retinal, retinoic acid, retinol esters), vitamin D (calciferol, cholecalciferol, vitamin D₃, 1,25,-dihydroxyvitamin D), vitamin E (α-tocopherol, α-tocopherol acetate, α-tocopherol succinate, α-tocopherol nicotinate, α-tocopherol), vitamin K (vitamin K₁, phylloquinone, naphthoquinone, vitamin K₂, menaquinone-7, vitamin K₃, menaquinone-4, menadione, menaquinone-8, menaquinone-8H, menaquinone-9, menaquinone-9H, menaquinone-10, menaquinone-11, menaquinone-12, menaquinone-13), choline, inositol, β-carotene and any combinations thereof.

In embodiments providing a children's nutritional product, such as a growing-up milk, the composition may optionally include, but is not limited to, one or more of the following minerals or derivations thereof: boron, calcium, calcium acetate, calcium gluconate, calcium chloride, calcium lactate, calcium phosphate, calcium sulfate, chloride, chromium, chromium chloride, chromium picolonate, copper, copper sulfate, copper gluconate, cupric sulfate, fluoride, iron, carbonyl iron, ferric iron, ferrous fumarate, ferric orthophosphate, iron trituration, polysaccharide iron, iodide, iodine, magnesium, magnesium carbonate, magnesium hydroxide, magnesium oxide, magnesium stearate, magnesium sulfate, manganese, molybdenum, phosphorus, potassium, potassium phosphate, potassium iodide, potassium chloride, potassium acetate, selenium, sulfur, sodium, docusate sodium, sodium chloride, sodium selenate, sodium molybdate, zinc, zinc oxide, zinc sulfate and mixtures thereof. Non-limiting exemplary derivatives of mineral compounds include salts, alkaline salts, esters and chelates of any mineral compound.

The minerals can be added to growing-up milks or to other children's nutritional compositions in the form of salts such as calcium phosphate, calcium glycerol phosphate, sodium citrate, potassium chloride, potassium phosphate, magnesium phosphate, ferrous sulfate, zinc sulfate, cupric sulfate, manganese sulfate, and sodium selenite. Additional vitamins and minerals can be added as known within the art.

The nutritional compositions of the present disclosure may optionally include one or more of the following flavoring agents, including, but not limited to, flavored extracts, volatile oils, cocoa or chocolate flavorings, peanut butter flavoring, cookie crumbs, vanilla or any commercially available flavoring. Examples of useful flavorings include, but are not limited to, pure anise extract, imitation banana extract, imitation cherry extract, chocolate extract, pure lemon extract, pure orange extract, pure peppermint extract, honey, imitation pineapple extract, imitation rum extract, imitation strawberry extract, or vanilla extract; or volatile oils, such as balm oil, bay oil, bergamot oil, cedarwood oil, cherry oil, cinnamon oil, clove oil, or peppermint oil; peanut butter, chocolate flavoring, vanilla cookie crumb, butterscotch, toffee, and mixtures thereof. The amounts of flavoring agent can vary greatly depending upon the flavoring agent used. The type and amount of flavoring agent can be selected as is known in the art.

The nutritional compositions of the present disclosure may optionally include one or more emulsifiers that may be added for stability of the final product. Examples of suitable emulsifiers include, but are not limited to, lecithin (e.g., from egg or soy), alpha lactalbumin and/or mono- and di-glycerides, and mixtures thereof. Other emulsifiers are readily apparent to the skilled artisan and selection of suitable emulsifier(s) will depend, in part, upon the formulation and final product.

The nutritional compositions of the present disclosure may optionally include one or more preservatives that may also be added to extend product shelf life. Suitable preservatives include, but are not limited to, potassium sorbate, sodium sorbate, potassium benzoate, sodium benzoate, calcium disodium EDTA, and mixtures thereof.

The nutritional compositions of the present disclosure may optionally include one or more stabilizers. Suitable stabilizers for use in practicing the nutritional composition of the present disclosure include, but are not limited to, gum arabic, gum ghatti, gum karaya, gum tragacanth, agar, furcellaran, guar gum, gellan gum, locust bean gum, pectin, low methoxyl pectin, gelatin, microcrystalline cellulose, CMC (sodium carboxymethylcellulose), methylcellulose hydroxypropyl methyl cellulose, hydroxypropyl cellulose, DATEM (diacetyl tartaric acid esters of mono- and diglycerides), dextran, carrageenans, and mixtures thereof.

The nutritional compositions of the disclosure may provide minimal, partial or total nutritional support. The compositions may be nutritional supplements or meal replacements. The compositions may, but need not, be nutritionally complete. In an embodiment, the nutritional composition of the disclosure is nutritionally complete and contains suitable types and amounts of lipid, carbohydrate, protein, vitamins and minerals. The amount of lipid or fat typically can vary from about 1 to about 25 g/100 kcal. The amount of protein typically can vary from about 1 to about 7 g/100 kcal. The amount of carbohydrate typically can vary from about 6 to about 22 g/100 kcal.

In an embodiment, the children's nutritional composition may contain between about 10 and about 50% of the maximum dietary recommendation for any given country, or between about 10 and about 50% of the average dietary recommendation for a group of countries, per serving of vitamins A, C, and E, zinc, iron, iodine, selenium, and choline. In another embodiment, the children's nutritional composition may supply about 10-30% of the maximum dietary recommendation for any given country, or about 10-30% of the average dietary recommendation for a group of countries, per serving of B-vitamins. In yet another embodiment, the levels of vitamin D, calcium, magnesium, phosphorus, and potassium in the children's nutritional product may correspond with the average levels found in milk. In other embodiments, other nutrients in the children's nutritional composition may be present at about 20% of the maximum dietary recommendation for any given country, or about 20% of the average dietary recommendation for a group of countries, per serving.

In some embodiments the nutritional composition is an infant formula. Infant formulas are fortified nutritional compositions for an infant. The content of an infant formula is dictated by federal regulations, which define macronutrient, vitamin, mineral, and other ingredient levels in an effort to simulate the nutritional and other properties of human breast milk. Infant formulas are designed to support overall health and development in a pediatric human subject, such as an infant or a child.

In some embodiments, the nutritional composition of the present disclosure is a growing-up milk. Growing-up milks are fortified milk-based beverages intended for children over 1 year of age (typically from 1-3 years of age, from 4-6 years of age or from 1-6 years of age). They are not medical foods and are not intended as a meal replacement or a supplement to address a particular nutritional deficiency. Instead, growing-up milks are designed with the intent to serve as a complement to a diverse diet to provide additional insurance that a child achieves continual, daily intake of all essential vitamins and minerals, macronutrients plus additional functional dietary components, such as non-essential nutrients that have purported health-promoting properties.

The exact composition of a growing-up milk or other nutritional composition according to the present disclosure can vary from market-to-market, depending on local regulations and dietary intake information of the population of interest. In some embodiments, nutritional compositions according to the disclosure consist of a milk protein source, such as whole or skim milk, plus added sugar and sweeteners to achieve desired sensory properties, and added vitamins and minerals. The fat composition may, in some embodiments, include an enriched lipid fraction derived from milk. Total protein can be targeted to match that of human milk, cow milk or a lower value. Total carbohydrate is usually targeted to provide as little added sugar, such as sucrose or fructose, as possible to achieve an acceptable taste. Typically, Vitamin A, calcium and Vitamin D are added at levels to match the nutrient contribution of regional cow milk. Otherwise, in some embodiments, vitamins and minerals can be added at levels that provide approximately 20% of the dietary reference intake (DRI) or 20% of the Daily Value (DV) per serving. Moreover, nutrient values can vary between markets depending on the identified nutritional needs of the intended population, raw material contributions and regional regulations.

The disclosed nutritional composition(s) may be provided in any form known in the art, such as a powder, a gel, a suspension, a paste, a solid, a liquid, a liquid concentrate, a reconstituteable powdered milk substitute or a ready-to-use product. The nutritional composition may, in certain embodiments, comprise a nutritional supplement, children's nutritional product, infant formula, human milk fortifier, growing-up milk or any other nutritional composition designed for an infant or a pediatric subject. Nutritional compositions of the present disclosure include, for example, orally-ingestible, health-promoting substances including, for example, foods, beverages, tablets, capsules and powders. Moreover, the nutritional composition of the present disclosure may be standardized to a specific caloric content, it may be provided as a ready-to-use product, or it may be provided in a concentrated form. In some embodiments, the nutritional composition is in powder form with a particle size in the range of 5 μm to 1500 μm, more preferably in the range of 10 μm to 300 μm.

All combinations of method or process steps as used herein can be performed in any order, unless otherwise specified or clearly implied to the contrary by the context in which the referenced combination is made.

The methods and compositions of the present disclosure, including components thereof, can comprise, consist of, or consist essentially of the essential elements and limitations of the embodiments described herein, as well as any additional or optional ingredients, components or limitations described herein or otherwise useful in nutritional compositions.

### EXAMPLES

### Example 1

Example 1 describes the microbiome changes in fecal matter of laboratory rats fed diets of PDX and GOS, LGG, and both PDX, GOS, and LGG as compared to a control.

Briefly, weanling (postnatal day 21) Long Evans (LE) rats were fed control or PDX/GOS diet chow (GOS 7 g/kg+PDX 7 g/kg) for four weeks. Probiotic LGG was reconstituted at a concentration of 1×10⁸ CFU/ml in drinking water. Each cage received between 80-150 mL each day depending on size and number of animals per cage. Each rat was randomly assigned across the treatment groups. Animals were maintained on a 12/12 light/dark cycles. The memory test, assessed using the time-dependent version of novel object recognition, was performed during the animal's light cycle phase. Body weights were taken three times a week for the duration of the study. Observations of any clinical signs of illness were noted. Food consumption was measured every other day for the duration of the study. Fecal samples were collected at three time points (baseline, day of treatment introduction, and the end of the experiment. Across the time points, samples were collected approximately the same time of day. Care was taken to avoid cross contamination samples across treatment groups.

The microbiome analysis of the fecal samples included the diversity examined from two perspectives. First, overall richness (i.e., number of distinct organisms present with the microbiome), was expressed as the number of operational taxonomic units (OTUs), with OTUs being defined as sequence clusters that were 97% similar. Second, overall diversity (which is determined by both richness and evenness, the distribution of abundance among distinct taxa) was expressed as Shannon Diversity. Shannon diversity (H′) is calculated via the following:

$$H = - {\sum }_{i = 1}^{R} {p}_{i} log {p}_{i}$$

Where R is richness and pᵢ is the relative abundance of the ith OTU. For both, rarefaction was used to indicate the impact of sampling depth on diversity.

Individual bacterial taxa were screened for group differences using a mixed-effects ANOVA (the random effect was used to account for multiple observations from the same subject). Prior to analysis, relative abundances were transformed using an arcsin transformation. P-values were adjusted to maintain a false discovery rate (FDR) of 5%.

As a more direct analysis, individual OTUs also were examined for significant changes over time. Here, each treatment group was considered separately, and OTU count data were analyzed. Within subject variability was removed prior to testing for a significant change over time.

Multivariate differences among groups were evaluated with “Permutational Multivariate Analysis of Variance Using Distance Matrices” function adonis. For the ADONIS analysis, distances among samples first were calculated using UniFrac or Bray-Curtis distances, and then an ANOVA-like simulation was conducted to test for group differences.

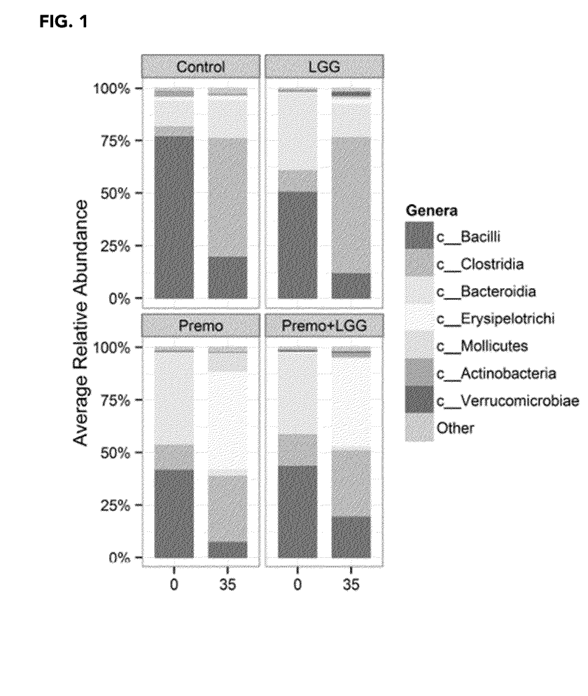

Our results showed reduced Clostridia in rats fed PDX/GOS diet on day 35 (FIG. 1). Generally, breast-fed infants were shown to have lower ratio of Clostridia compared to formula-fed infants (Azad 2013). Thus lower levels of Clostridia seen in PDX/GOS animals might be beneficial for overall health of the animal.

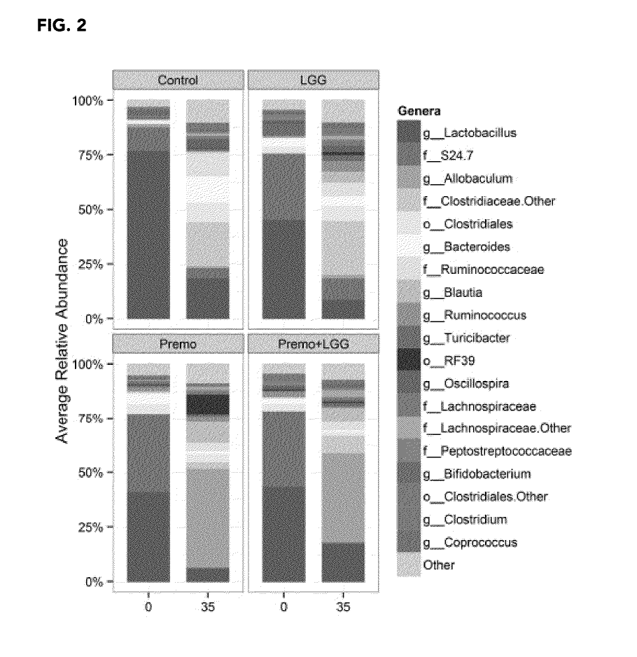

At the genus level, as shown in FIG. 2, PDX/GOS and PDX/GOS+LGG treatments increased genus Allobacullum, which is a lactic acid and a butyric acid producer (Greetham 2004). These products can contribute to decrease of the stool pH, as seen in breast-fed infants. Overall, in formula-fed infants, the stool pH is higher compared to breast-fed infants. In addition, Allobaculum may provide additional cognitive benefits via the gut-brain-axis pathways.

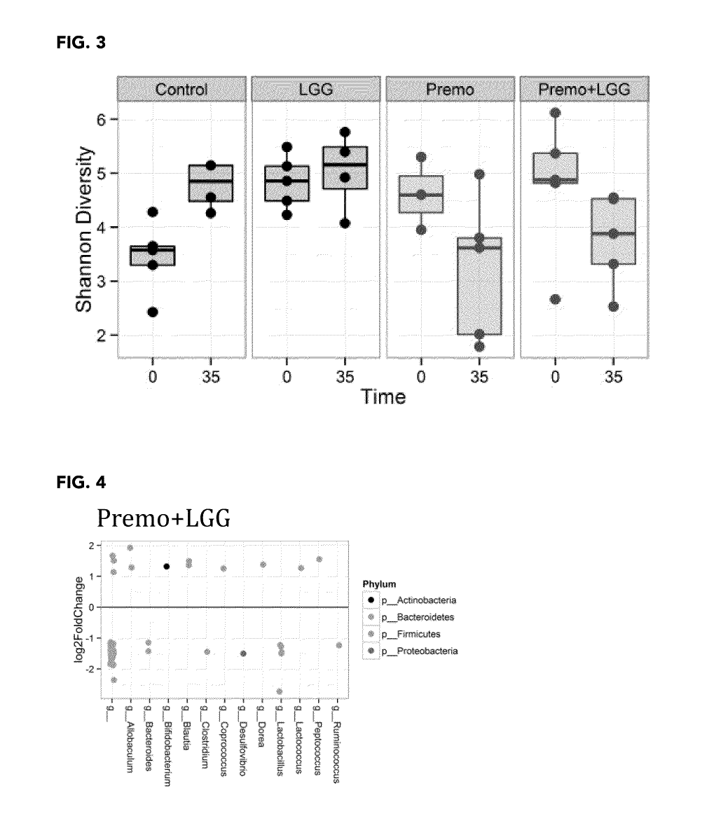

Moreover the microbiome analysis revealed that PDX/GOS and PDX/GOS+LGG treatments decrease bacterial diversity over time (FIG. 3). Lower bacterial diversity is generally observed in breast-fed infants compared to formula-fed infants. For example, in the study by Azad et al. (2013) formula fed infants had increased richness of species. Thus PDX/GOS diet could decrease the richness of species similarly to breast-fed infants.

As shown in FIG. 4, there was an increase in phylum Actinobacteria in the PDX/GOS+LGG group. Previous studies have demonstrated that Actinobacteria level is higher in breast-fed infants compared to formula-fed infants (Harmsen 2000). Thus increasing levels of Actinobacteria might have benefits in formula-fed infants.

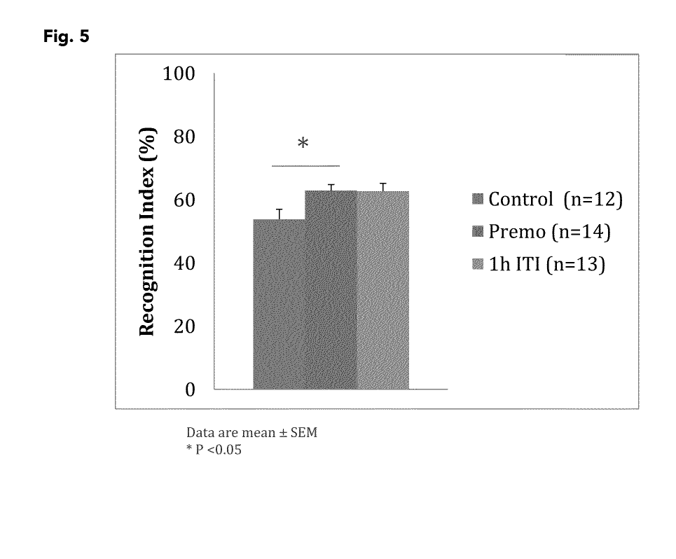

The novel object recognition test revealed that PDX/GOS fed LE rats had a significantly higher recognition index than rats fed control diet (P<0.05). Body weight, water and food intake did not differ between the diet groups (FIG. 5).

Accordingly, there were changes in the fecal microbiota over time in control groups. The PDX/GOS group resulted in driving significant modulation of rodent microbiota. Further, the change in microbiota composition may have an effect on behavior as there was an increase in Allobaculum, which is a short chain fatty acid producer.

### Example 2

This example describes the effect of LGG on the neurotransmitter levels in the brain. Briefly, chronic visceral hyperalgesia was induced in rats by administration of intracolonic zymosan (or normal saline for control) for three consecutive days during postnatal day 14-16 (P14-P16). LGG treatment was initiated after weaning (P21) and continued until P60. The levels of neurotransmitters and amino acids were quantified in the frontal cortex, sub-cortex, brain stem and cerebellum.

The quantitative assessment of neurotransmitters was conducted using HPLC-based separation followed by fluorescent and/or electrochemical detection. Briefly, brain sections were homogenized, using a tissue dismembrator, in 100-750 ul of 0.1 M TCA, which contains 10-2 M sodium acetate, 10-4 M EDTA, 5 ng/ml isoproterenol (as internal standard) and 10.5% methanol (pH 3.8). Samples were spun in a microcentrifuge at 10000 g for 20 minutes. Samples of the supernatant were then analyzed for neurotransmitters (biogenic monoamines). Biogenic amines were determined by a specific HPLC assay utilizing an Antec Decade II (oxidation: 0.4) (3 mm GC WE, HYREF) electrochemical detector operated at 33° C. Twenty I samples of the supernatant were injected using a Water 2707 autosampler onto a Phenomenex Kintex (2.6u, 100A) C18 HPLC column (100×4.60 mm). Biogenic amines were eluted with a mobile phase consisting of 89.5% 0.1 M TCA, 10-2 M sodium acetate, 10-4 M EDTA and 10.5% methanol (pH 3.8). Solvent was delivered at 0.6 ml/min using a Waters 515 HPLC pump. Using this HPLC solvent the following biogenic amines eluted in the following order: noradrenaline, Adrenaline, DOPAC, Dopamine, 5-HIAA, HVA, 5-HT, and 3-MT. HPLC control and data acquisition were managed by Empower software.

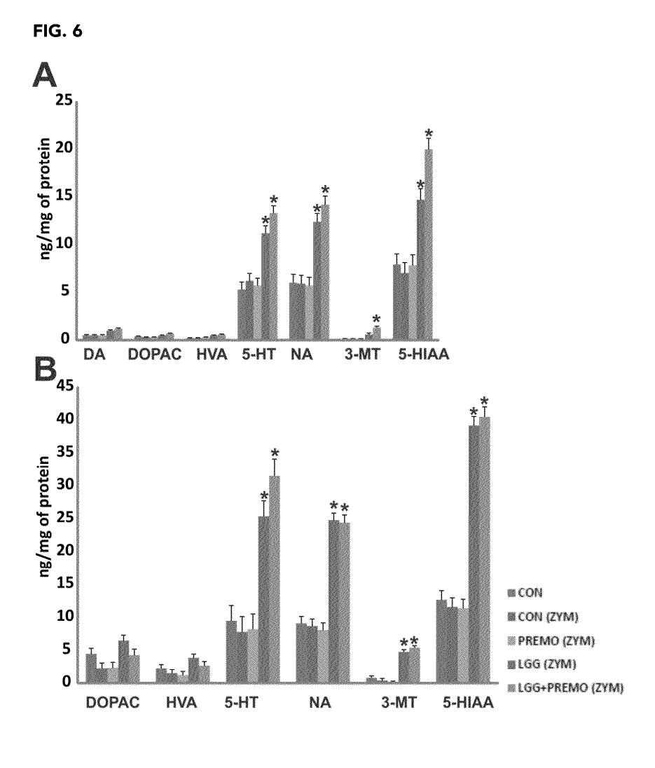

FIGS. 6A and 6B illustrate the effect of LGG treatment on the levels of neurotransmitters in the brain stem and subcortex of control and experimental rats. In the brain stem (FIG. 6A), LGG treatment produced a significant increase in the levels of serotonin (5-HT), 5-hydroxyindoleacetic acid (5-HIAA), noradrenaline (NA) and metallothionin (3-MT) compared to non-LGG treated rats. A similar effect in the levels of neurotransmitters was observed in the sub-cortex of rats treated with LGG (FIG. 6B). 5-HT and NA play role in spinal descending inhibition of pain. 5-HT is present in the central and peripheral serotonergic neurons, it is released from platelets and mast cells after tissue injury, and it exerts algesic and analgesic effects depending on the site of action and the receptor subtype (Sommer, 2004). Similarly, NA is generally reported to alter pain behavior by its action on spinal α2-adrenoreceptors. There is evidence, however, that NA acting through α2-adrenoreceptors has anti-nociceptive effects by acting both at spinal and supraspinal sites including in the locus coeruleus (Pertovaara et al., 1991). Overall, LGG has a profound impact on the levels of neurotransmitters in the brain, which might in turn be responsible for the neonatal zymosan-treated rats not exhibiting visceral hyperalgesia following LGG treatment.

These results highlight the potential role of LGG in the bidirectional communication of the gut-brain axis and suggest that LGG may be a useful therapeutic option in treating chronic visceral pain in neonates.

This study shows for the first time the direct effect of LGG in modulating the visceral nociception via altering the levels of several key neurotransmitters in CNS that are involved in pain perception.

### Example 3

Example 3 shows the efficacy of LGG treatment in reducing visceral pain sensitivity.

Example 3 utilized a rat colonic zymosan-treated hyperalgesia model (i.e. a model of post-inflammatory visceral pain sensitivity). Zymosan was injected into the colon during the neonatal period producing short-term inflammation and subsequent long-term colonic hypersensitivity. The data demonstrated that LGG attenuated visceral hypersensitivity.

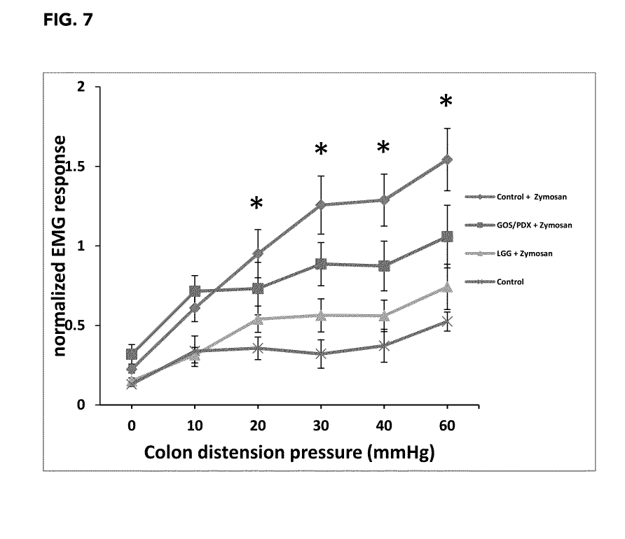

As can be seen in FIG. 7, neonatal intra-colonic zymosan instillation produced visceral hyperalgesia in adult rats as observed by significant increase in viscera-motor response (VMR) as compared to colorectal distension (CRD) compared to intra-colonic saline-treated rats (Control). As can be further seen in FIG. 7, treatment with LGG significantly attenuated the viscera-motor response. Thus, as shown in FIG. 7, LGG, GOS, and PDX had a significant visceral analgesic effect in zymosan-induced colitis. The introduction of zymosan produced visceral hyperalgesia in adult rats as observed by significant increase of electromyography (EMG) recordings (*p<0.05 vs Control). Treatment with probiotic LGG or GOS/PDX significantly attenuated the sensitivity to pain (p<0.05 vs Control+Zymosan; n=10)

In this experiment, weanling rats were fed a control diet or control diet plus LGG and/or PDX/GOS for 40 days. The VMR to CRD was quantified using electromyographic (EMG) recordings from the external oblique muscle of the abdomen as an objective measure of visceral sensation in all groups. A stimulus-response function to graded CRD was constructed to test the colonic intensity dependent increase in EMG activity change of external oblique muscle.

Formulation examples are provided to illustrate some embodiments of the nutritional composition of the present disclosure but should not be interpreted as any limitation thereon. Other embodiments within the scope of the claims herein will be apparent to one skilled in the art from the consideration of the specification or practice of the nutritional composition or methods disclosed herein. It is intended that the specification, together with all the examples disclosed herein, be considered to be exemplary only, with the scope and spirit of the disclosure being indicated by the claims, which follow the examples.

### FORMULATION EXAMPLES

TABLE 1
Nutritional composition including LGG, GOS, and PDX.

| | per 100 kcal | |
|---|---|---|
| Nutrient/Lipid | Minimum | Maximum |
| Protein (g) | 1 | 7 |
| Fat (g) | 1 | 10 |
| Carbohydrates (g) | 5 | 25 |
| DHA (mg) | 5 | 100 |
| GOS (g) | 0.1 | 1.0 |
| PDX (g) | 0.1 | 0.5 |
| LGG (CFU) | 1 × 10⁴ | 1.5 × 10¹⁰ |
| Vitamin A (IU) | 134 | 921 |
| Vitamin D (IU) | 22 | 126 |
| Vitamin E (IU) | 0.8 | 5.4 |
| Vitamin K (mcg) | 2.9 | 18 |
| Thiamin (mcg) | 63 | 328 |
| Riboflavin (mcg) | 68 | 420 |
| Vitamin B6 (mcg) | 52 | 397 |
| Vitamin B12 (mcg) | 0.2 | 0.9 |
| Niacin (mcg) | 690 | 5881 |
| Folic acid (mcg) | 8 | 66 |
| Panthothenic acid (mcg) | 232 | 1211 |
| Biotin (mcg) | 1.4 | 5.5 |
| Vitamin C (mg) | 4.9 | 24 |
| Choline (mg) | 4.9 | 43 |
| Calcium (mg) | 68 | 297 |
| Phosphorus (mg) | 54 | 210 |
| Magnesium (mg) | 4.9 | 34 |
| Sodium (mg) | 24 | 88 |
| Potassium (mg) | 82 | 346 |
| Chloride (mg) | 53 | 237 |
| Iodine (mcg) | 8.9 | 79 |
| Iron (mg) | 0.7 | 2.8 |
| Zinc (mg) | 0.7 | 2.4 |
| Manganese (mcg) | 7.2 | 41 |
| Copper (mcg) | 16 | 331 |

All references cited in this specification, including without limitation, all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinence of the cited references.

Although embodiments of the disclosure have been described using specific terms, devices, and methods, such description is for illustrative purposes only. The words used are words of description rather than of limitation. It is to be understood that changes and variations may be made by those of ordinary skill in the art without departing from the spirit or the scope of the present disclosure, which is set forth in the following claims. In addition, it should be understood that aspects of the various embodiments may be interchanged in whole or in part. Therefore, the spirit and scope of the appended claims should not be limited to the description of the versions contained therein.

What is claimed is:

1. A method for reducing the risk of visceral pain hypersensitivity and reducing the functional abdominal pain and infantile colic in a pediatric subject comprising providing to the pediatric subject a nutritional composition comprising, a carbohydrate source, a protein source, a fat source, from about 1×10⁴ CFU/100 kcal to about 1.5×10¹⁰ CFU/100 kcal of Lactobacillus rhamnosus GG, from about 0.1 g/100 kcal to about 0.5 g/100 kcal of polydextrose, and from about 0.1 g/100 kcal to about 1.0 g/100 kcal of galacto-oligosaccharides.

2. The method of claim 1, wherein the nutritional composition further comprises lactoferrin.

3. The method of claim 1, wherein the nutritional composition further comprises docosahexaenoic acid.

4. The method of claim 3, wherein the nutritional composition further comprise arachidonic acid.

5. The method of claim 4, the weight ratio of docosahexaenoic acid to arachidonic acid is from about 1:3 to about 9:1.

6. The method of claim 1, wherein the nutritional composition comprises from about 1×10⁵ cfu/100 kcals to about 1.5×10⁹ cfu/100 kcals of Lactobacillus rhamnosus GG.

7. The method of claim 1, wherein the nutritional composition further comprises β-glucan.

8. The method of claim 1, wherein the nutritional composition further comprises a source of iron.

9. The method of claim 1, wherein the nutritional composition is an infant formula.

10. A method for modifying the gut-brain axis in a pediatric subject by providing a nutritional composition comprising per 100 kcal:
(i) between about 6 g and about 22 g of a carbohydrate source;
(ii) between about 1 g and about 7 g of a protein source;
(iii) between about 1 g and about 10.3 g of a fat source;
(iv) between about 1×10⁴ CFU and 1.5×10¹⁰ CFU of Lactobacillus rhamnosus GG;
(v) between about 0.1 g and 1.0 g of galacto-oligosaccharides; and
(vi) between about 0.1 g and 0.5 g of polydextrose;
to the pediatric subject.

11. The method of claim 10, wherein the pediatric subject is an infant.

12. The method of claim 10, wherein the nutritional composition is an infant formula.

13. The method of claim 10, wherein the nutritional composition further comprises a culture supernatant.

14. The method of claim 10, wherein the nutritional composition further comprises β-glucan.

15. The method of claim 10, wherein the nutritional composition further comprises a source of iron.

16. A method of reducing the functional abdominal pain in a pediatric subject by providing a nutritional composition comprising a carbohydrate source, a protein source, a fat source, from about 1×10⁴ CFU/100 kcal to about 1.5×10¹⁰ CFU/100 kcal of Lactobacillus rhamnosus GG, from about 0.1 mg/100 kcal to about 0.5 mg/100 kcal of polydextrose, and from about 0.015 mg/100 kcal to about 1.5 mg/100 kcal of galacto-oligosaccharides.

17. The method of claim 16, wherein the nutritional composition further comprises cholesterol.

18. The method of claim 16, wherein the nutritional composition further comprise DHA.

19. The method of claim 16, wherein the nutritional composition further comprises ARA.

20. The method of claim 16, wherein the nutritional composition is an infant formula.
